(19) United States
(12) Patent Application Publication
(10) Pub. No.: US 2022/0338807 A1
(43) Pub. Date: Oct. 27, 2022

(54) TRIAL INSTRUMENTS FOR LIGATURE RECONSTRUCTION

(71) Applicant: THINK SURGICAL, INC., Fremont, CA (US)

(72) Inventor: Timothy J. Pack, Fremont, CA (US)

(73) Assignee: THINK SURGICAL, INC., Fremont, CA (US)

(21) Appl. No.: 17/620,833

(22) PCT Filed: Jun. 19, 2020
(86) PCT No.: PCT/US2020/038652

§ 371 Date: Dec. 20, 2021

Related U.S. Application Data
(60) Provisional application No. 62/863,605, filed on Jun. 19, 2019.

(51) Int. Cl. A61B 5/00; A61B 5/11

(52) U.S. Cl. CPC A61B 5/4851; A61B 5/1121; A61B 5/1127; A61B 5/4533

(57) ABSTRACT

A device, method, and system are provided for measuring the fit and mechanical properties of an intended ligament or tendon graft during a surgical procedure. The device includes a sensor body, a set of temporary fixation securements that terminate a set of opposing sides of the sensor body, and electronic circuitry in communication with the sensor body, where the electronic circuitry generates electric signals in proportion to the mechanical properties experienced by the sensor body. The set of temporary fixation securements are adapted to attach to at least one of: (i) one or more patient bones, or (ii) permanent fixation hardware assembled to one or more patient bones.

### RELATED APPLICATIONS

This application claims priority benefit of U.S. Provisional Application Ser. No. 62/863,605 filed 19 Jun. 2019, the contents of which are hereby incorporated by reference.

### TECHNICAL FIELD

The present invention generally relates to ligament reconstruction surgery, and in particular to systems and methods for measuring the fit, and mechanical properties of an intended ligament graft prior to implantation of the actual ligament during a surgical procedure.

### BACKGROUND

Rupture of the anterior cruciate ligament (ACL) is one of the most frequent injuries to the knee joint. ACL reconstruction is a major orthopedic procedure most often performed to repair the knee joint. Early stabilization of the knee joint by ACL reconstruction also decreases the risk of injury to other important structures.

The goal of anterior cruciate ligament (ACL) reconstruction procedures, as well as other ligament and tendon repairs to joints including the elbow, is to replace a ruptured ligament or tendon with a graft that is intended to provide similar mechanical stability relative to the native anatomy while preserving the range of motion of the knee or joint. However, the native cruciate ligature of the knee is highly complex, and presents several challenges for successful reconstruction procedures.

During ACL reconstruction procedures a graft is placed into roughly the same location that the native ACL occupied prior to rupture. To achieve this colocation with a graft, holes are drilled in the femur and tibia in the approximate footprint of the native ACL. A graft is placed in these tunnels, and fixated by some means on both ends. The intent of the graft is to restore stability to the injured knee, while maintaining range of motion. However, the biggest challenge in ACL reconstruction is typically the exact placement of drilled bone tunnels. When poorly placed, bone tunnels significantly affect the outcome of surgery. Outcomes affected by poor tunnel placement include restricted range of motion, knee joint instability, reaction of the synovium in the knee, and knee joint pain. Furthermore, impingement of the graft and/or improper graft tension may result in potential graft failure with lesion development. Currently, manual testing is the only way to check graft properties, and manual testing is highly subjective.

Precisely placed bone tunnels are difficult to achieve through current surgical methods. While ACL reconstruction is predominately performed arthroscopically, arthroscopy does not allow the surgeon to gain a complete 3D view of important anatomical structures, particularly in the anteroposterior direction. Large incisions are often required to provide surgeons adequate access to landmarks and/or drill angles. Further, as ACL reconstructions require a high learning curve to master, attainable only from high volumes and extensive experience, ACL reconstructions are most often performed by under experienced orthopedic surgeons. It is estimated that up to 20% of ACL grafts fail due to impingement, improper graft tension, or poor tunnel placement.

Various techniques have been developed to help a surgeon correctly plan and create bone tunnels for implantation and attachment of ligaments to the bones of a joint. One system and method to optimize ligament reconstruction surgical outcomes is achieved by enabling bone tunnels to be precisely and optimally placed through the use of pre-operative planning systems coupled with precision control bone evacuation machines, such as robotic drills is described in U.S. Pat. No. 10,034,675, assigned to the assignee of the present application, the contents of which is incorporated herein in its entirety by reference.

While there have been advancements in ligament replacement and reconstructive surgeries, the highly subjective manual test for checking graft properties is still sub optimal. Thus, there exists a need for a system and non-subjective method for measuring the fit, and mechanical properties of an intended ligament or tendon graft during a surgical procedure.

### SUMMARY OF THE INVENTION

A device is provided for measuring mechanical properties of an intended ligament or tendon graft during a surgical procedure. The device includes a sensor body, a set of temporary fixation securements that terminate a set of opposing sides of the sensor body, and electronic circuitry in communication with the sensor body, where the electronic circuitry generates electric signals in proportion to the mechanical properties experienced by the sensor body. The set of temporary fixation securements are adapted to attach to at least one of: (i) one or more patient bones, or (ii) permanent fixation hardware assembled to one or more patient bones.

A method is provided for measuring mechanical properties of an intended ligament or tendon graft during a surgical procedure. The method includes placing the sensor body described above in an intended position of a planned graft, securing the sensor body with a set of temporary fixation securements to at least one of one or more patient bones that form a movable joint. The movable joint of the joint bridged relative to the sensor body is then exercised to obtain measurements of the mechanical properties the sensor body is experiencing in response to exercising the movable joint, and determining whether the sensor body is improperly tensioned, or experiencing impingement based on the obtained measurements.

A computer-assisted surgical system is provided that includes the device for measuring mechanical properties of an intended ligament or tendon graft as described, one or more computers with software, and a display to display the output from the one or more computers in real-time. The one or more computers receiving and processing the electric signals generated by the electronic circuitry in proportion to the mechanical forces experienced by the sensor body.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 illustrates a prior art bonded foil strain gauge that may be used in embodiments of the inventive ligament/tendon trial implant device;

FIG. 2 shows a prior art strain gauge diagram formed as a Wheatstone bridge and an equivalent mathematical model;

FIG. 3 illustrates a ligament/tendon trial implant graft inserted in an intended position of a permanent anterior cruciate ligament (ACL) reconstructive graft in accordance with embodiments of the invention;

FIG. 4 illustrates a ligament/tendon trail implant graft having a double bundle and external leads in an intended position of a permanent ACL reconstructive graft in accordance with embodiments of the invention;

FIG. 5 depicts a method for measuring the fit, and mechanical properties of an intended ligament or tendon graft using a ligament/tendon trial implant graft such as that of FIG. 3 during a surgical procedure in accordance with embodiments of the invention; and

FIG. 6 depicts a surgical system in the context of an operating room (OR) with a surgical robot for implementing the method of FIG. 5 in accordance with embodiments of the invention.

### DETAILED DESCRIPTION

The present invention has utility as a system and method for measuring the fit, and mechanical properties of an intended ligament or tendon graft during a surgical procedure. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Further, it should be appreciated that although the systems and methods described herein make reference to anterior cruciate ligament (ACL) reconstruction procedures, the systems and methods may be applied to other surgical procedures involving other ligatures and tendons involved with joints in the body illustratively including the hip, ankle, elbow, wrist, as well as revision of initial repair or replacement of any joints.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “digitizer” refers to a measuring device capable of measuring physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; or an end-effector of a robotic device.

As used herein, the term “digitizing” refers to the collecting, measuring, and/or recording of physical points in space with a digitizer.

As used herein, the term “pre-operative bone data” refers to bone data used to pre-operatively plan a procedure before making modifications to the actual bone. The pre-operative bone data may include one or more of the following. An image data set of a bone (e.g., computed tomography, magnetic resonance imaging, ultrasound, x-ray, laser scan), a virtual generic bone model, a physical bone model, a virtual patient-specific bone model generated from an image data set of a bone, or a set of data collected directly on a bone intra-operatively commonly used with imageless computer-assist devices.

Also described herein are “computer-assisted surgical systems.” A computer assisted surgical system refers to any system requiring a computer to aid in a surgical procedure. Examples of computer-assisted surgical systems include 1-N degree of freedom hand-held surgical systems, tracking systems, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401; 7,206,626; 8,876,830; 8,961,536; and 9,707,043; and PCT Publication WO2017/058520. In particular inventive embodiments, the surgical system is a robotic surgical system as described below. In particular inventive embodiments, the surgical system is a 2-DOF articulating device as described in PCT Publication WO2017/091380. The surgical system may provide autonomous, semi-autonomous, or haptic control and any combinations thereof. In addition, a user may manually maneuver a tool attached to the surgical system while the system provides at least one of power, active, or haptic control to the tool.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a tracking marker array) associated with the bone, if such landmarks exist. Methods of registration known in the art are described in U.S. Pat. Nos. 6,033,415, 8,010,177, and 8,287,522.

Also, referenced herein is a surgical plan. For context, a surgical plan is created, either pre-operatively or intra-operatively, by a user using planning software. The planning software may be used to plan the position for an implant relative to pre-operative bone data. For example, the planning software may be used to generate three-dimensional (3-D) models of the patient's bony anatomy from a computed tomography (CT), magnetic resonance imaging (MRI), x-ray, ultrasound image data set, or from a set of points collected on the bone intra-operatively. A set of 3-D computer aided design (CAD) models of the manufacturer's prosthesis are pre-loaded in the software that allows the user to place the components of a desired prosthesis to the 3-D model of the boney anatomy to designate the best fit, position, and orientation of the implant to the bone.

As used herein, the term “real-time” refers to the processing of input data within milliseconds such that calculated values are available within 10 seconds of computational initiation.

Also used herein is the term “optical communication” which refers to wireless data transfer via infrared or visible light as described in U.S. Pat. No. 10,507,063 assigned to the assignee of the present application and incorporated by reference herein in its entirety.

Embodiments of the invention provide a ligament/tendon trial implant device that provides a way of measuring the fit, the mechanical properties, or both for an intended graft prior to the actual implantation of the permanent graft ligament or tendon. Embodiments of the trial graft devices are equipped with sensors to measure mechanical properties that the permanent graft will experience. Examples of sensors that may be used include stress/strain sensors. The measured mechanical properties are correlated to actual ligament and tendon performance, and provide the surgeon with several metrics to verify whether or not a given ligament or tendon replacement procedure will be successful. For example, sensors in the trial ligament provide the surgeon with data verifying whether or not the graft is improperly tensioned or is under impingement.

A strain gauge is a sensor whose resistance varies with applied force. A strain gauge converts force, pressure, tension, weight, etc., into a change in electrical resistance which can then be measured. When external forces are applied to a stationary object, stress and strain are the result. Stress is defined as the object's internal resisting forces, and strain is defined as the displacement and deformation that occur. Strain consists of tensile and compressive strain, distinguished by a positive or negative sign. Thus, strain gauges can be used to pick up expansion as well as contraction. The strain of a body is always caused by an external influence or an internal effect. Strain might be caused by forces, pressures, moments, heat, structural changes of the material and the like. If certain conditions are fulfilled, the amount or the value of the influencing quantity can be derived from the measured strain value. In experimental stress analysis this feature is widely used. Experimental stress analysis uses the strain values measured on the surface of a specimen, or structural part, to state the stress in the material and also to predict its safety and endurance. Special transducers can be designed for the measurement of forces or other derived quantities. These other properties that are illustratively measured include moments, pressures, accelerations, displacements, vibrations and others. A transducer generally contains a pressure sensitive diaphragm with strain gauges bonded thereto.

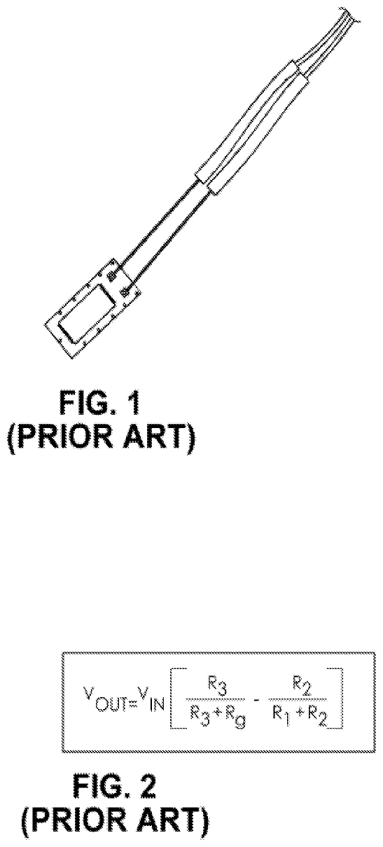

Referring now to the figures, FIG. 1 illustrates a typical bonded foil strain gauge as an example of a strain gauge that may be used in embodiments of the inventive ligament/tendon trial implant device. The metallic foil-type strain gauge includes of a grid of wire filament (a resistor) of approximately 0.001 in. (0.025 mm) thickness, bonded directly to the strained surface by a thin layer of epoxy resin. When a load is applied to the surface, the resulting change in surface length is communicated to the resistor and the corresponding strain is measured in terms of the electrical resistance of the foil wire, which varies linearly with strain. The foil diaphragm and the adhesive bonding agent work together in transmitting the strain, while the adhesive also serves as an electrical insulator between the foil grid and the surface.

In order to measure strain with a bonded resistance strain gauge, the strain gauge must be connected to an electric circuit that is capable of measuring the minute changes in resistance corresponding to strain. Strain gauge transducers usually employ four strain gauge elements that are electrically connected to form a Wheatstone bridge circuit. FIG. 2 shows a typical strain gauge diagram formed as a Wheatstone bridge, which is a divided bridge circuit used for the measurement of static or dynamic electrical resistance. The output voltage of the Wheatstone bridge is expressed in millivolts output per volt input. The relation between an input voltage V_{IN} to an output voltage Vₒᵤₜ is expressed by equation 1 (Eq. 1) also shown in FIG. 2. The number of active strain gauges that should be connected to the bridge depends on the application. For example, it may be useful to connect gauges that are on opposite sides of the ligature/tendon, one gauge in compression and the other gauge in tension. In this arrangement, the bridge output is effectively doubled for the same strain.

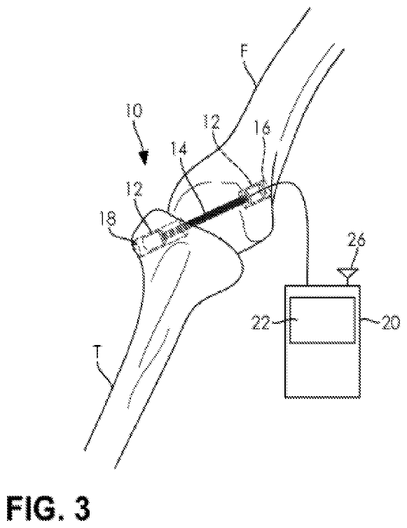

FIG. 3 illustrates an inventive embodiment of the ligament/tendon trial implant graft 10 inserted in the intended position of a permanent anterior cruciate ligament (ACL) reconstructive graft. Temporary fixation securements 12 are attached to the distal femur F and proximal tibia T bones that together form the knee joint within a drilled femoral tunnel 16 and tibia tunnel 18, respectively. A sensor body 14 that may a bonded foil strain gauge is modeled to have a form factor that mimics the intended ligature is connected to and terminated by the temporary fixation securements 12. The sensor body 14 may include a plurality of strain gauges, where each strain gauge is dedicated to measuring different outputs (e.g., one strain gauge measuring tension, and a second strain gauge measuring compression). One or more temporary fixation securements 12 may also house the electronic circuitry illustratively including the Wheatstone bridge to generate electric signals in proportion to the mechanical forces experienced by the ligament/tendon trial implant graft 10. The generated electrical signals may be transmitted via a wired 24 or wireless connection via antenna 26 to a computing device 20 equipped with an information display 22 that has a graphical user interface (GUI) that provides information to the surgeon as to whether or not the ligament/tendon trial implant graft 10 is improperly tensioned or is under impingement.

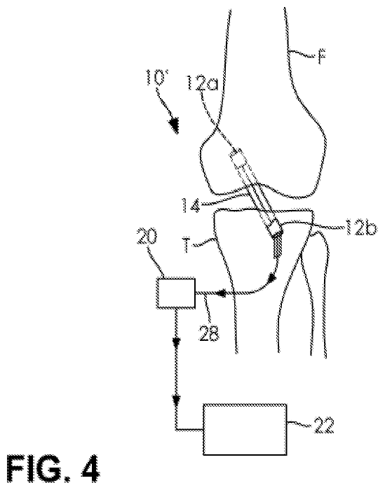

FIG. 4 depicts particular inventive embodiments of the ligament/tendon trial implant graft 10′ inserted in the intended position of a permanent anterior cruciate ligament (ACL) reconstructive graft. The ligament/tendon trial implant graft 10′ includes a temporary femoral fixation securement 12a, a temporary tibia fixation securement 12b, and a sensor body 14. The sensory body 14 may be an electrical strain gauge (e.g., bonded foil strain gauge) or a fiber optic strain gauge. Fiber optic strain gauges are particularly advantageous as they can be smaller, and embedded into biocompatible materials such as silicone. An example of a fiber optic strain gauge is the OSP-A & OSP-FT Fiber Optic Strain Gauge manufactured by Opsens Solutions Inc. headquartered in Québec, Québec, Canada. The sensor body 14 may further have a double bundle configuration to mimic the double bundles of a native ACL or a double bundled permanent ACL reconstructive graft. The double bundle configuration may include strain gauges (electrical or fiber optic) assembled together to form a double bundle. The double bundled sensor body 14 may further include one or more compression strain gauges dedicated to measuring any impingement or compression experienced on the trial graft 10′ throughout a range-of-motion of the knee joint. The compression strain gauge are in addition to the tension strain gauges, and may be located next to the tension strain gauges as part of the sensor body 14.

In particular embodiments, the electronic circuitry (e.g., transducers, Wheatstone Bridge) is not embedded as part of the sensor body 14 or temporary fixation elements 12. Data leads 28 from the strain gauges of the sensor body 14 may be exposed from the tibial end of the strain gauges since the tibial end of the trail graft 10 may remain outside of the body while trialing. The data leads 28 then connect to the electronic circuitry apart from the trail graft 10′. The electronic circuitry may be connected or housed in the computer device 20 to process the tension and/or compression experienced on the sensor body 14 in real-time. The tension and/or compression readings may be displayed in real-time on the display 22. This allows the user to put the patient through a full range of flexion/extension while monitoring the tension and/or compression in real-time.

In specific inventive embodiments, the temporary fixation elements 12 removably attach to permanent fixation hardware installed on the femur and tibia. The permanent fixation hardware may be the same hardware required to install the permanent ACL implant graft. The permanent fixation hardware may include a femoral anchor and a tibial anchor that are secured to the femur and tibia, respectively, prior to the attachment of the trail graft 10. The anchors may illustratively include a bone pin, a tack, a knot, percutaneous screws, or spiked washers. In particular embodiments, the femoral anchor is a percutaneous screw to secure the femoral end of the ligament/tendon trial implant graft 10 through a single hole, while the tibial anchor is a spiked washer assembled on the face of the tibia to anchor the tibial end of the ligament/tendon trial implant graft 10.

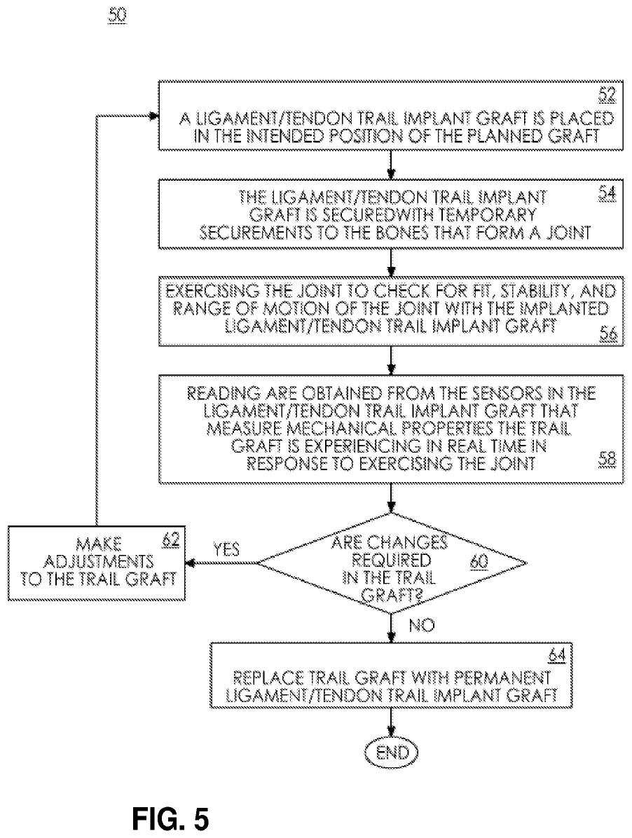

FIG. 5 depicts an embodiment of a method 50 for measuring the fit, and mechanical properties of an intended ligament or tendon graft during a surgical procedure. In embodiments of the invention, a surgeon places a ligament/tendon trial implant graft equipped with sensors in the intended position of the planned graft at Block 52. The ligament/tendon trial implant graft is secured with temporary securements to the bones that form a joint at Block 54. The surgeon subsequently flexes the joint to check for fit, stability, and range of motion of the joint with the implanted ligament/tendon trial implant graft at Block 56. Readings are obtained from the sensors in the ligament/tendon trial implant graft that measure mechanical properties the trial graft is experiencing in real time in response to exercising the joint at Block 58. The data provided by the sensors indicates to the surgeon whether or not the graft was improperly tensioned, or experiencing impingement, and the surgeon is able to determine if changes are required at Block 60. The surgeon may then adjust the size of the graft or fixation points until the mechanical measurements are satisfactory at Block 62. In a specific embodiment for an ACL replacement procedure, a tibial fixation apparatus is designed to allow for adjustment so that multiple lengths of the ligament/tendon trial implant graft may be tested. If additional adjustments to the trial graft are required (Block 62 is Yes) the steps performed in Blocks 52-60 are repeated on until satisfactory result for the trial graft are achieved and the permanent graft is implanted based on the results of the trial graft at Block 64.

Another method for measuring the fit, and mechanical properties of an intended ligament or tendon graft during a surgical procedure includes the following. Tibial permanent fixation hardware (tibial anchor) is installed on the tibia T and femoral permanent fixation hardware (femoral anchor) is installed on the femur. A surgeon places a ligament/tendon trial implant graft equipped with sensors in the intended position of the planned graft. The ligament/tendon trial implant graft is secured by attaching a femoral temporary securement on the graft to the femoral permanent fixation hardware, and attaching a tibial temporary securement on the graft to the tibial permanent fixation hardware. The surgeon subsequently flexes the joint to check for fit, stability, and range of motion of the joint with the implanted ligament/tendon trial implant graft. Readings are obtained from the sensors in the ligament/tendon trial implant graft that measure mechanical properties the trial graft is experiencing in real time in response to exercising the joint. The data provided by the sensors indicates to the surgeon whether or not the graft was improperly tensioned, or experiencing impingement, and the surgeon is able to determine if changes are required. The surgeon may then adjust the size of the graft or fixation points until the mechanical measurements are satisfactory. In a specific embodiment for an ACL replacement procedure, a tibial fixation apparatus is designed to allow for adjustment so that multiple lengths of the ligament/tendon trial implant graft may be tested. If additional adjustments to the trial graft are required, the locations of the permanent fixation hardware are adjusted and the trialing repeated until satisfactory result for the trial graft are achieved. Once achieved, the trial graft is removed while keeping the femoral permanent fixation hardware and tibial permanent fixation hardware in place on their respective bones. The permanent graft is then implanted using the femoral and tibial permanent fixation hardware as the fixation points for the permanent graft.

The femoral tunnel and tibia tunnel may be prepared using techniques known in the art. This includes the use of manual tool instrumentation to align a hand-held drill, such as the tools described in U.S. Pat. Nos. 4,257,411, 4,739,751, and 7,972,341. After the tunnels are prepared, the ligament/tendon trial implant graft may be used to measure the fit, and mechanical properties of an intended ligament or tendon graft during a surgical procedure as described above.

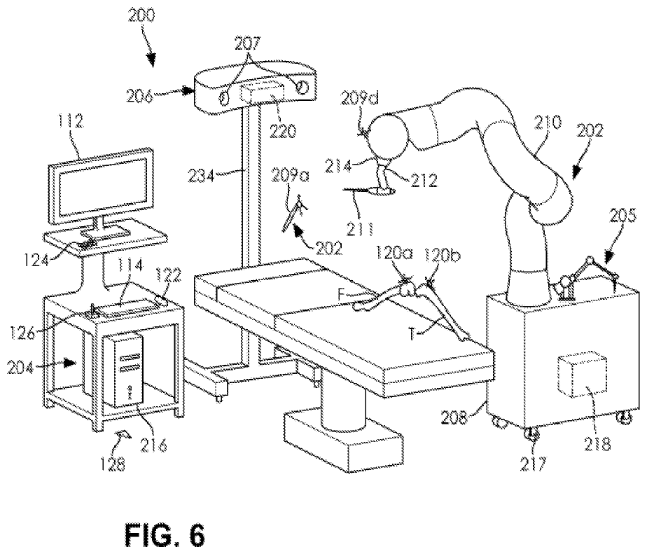

In specific embodiments, a computer-assisted surgical system is used to aid in the creation of the bone tunnels. FIG. 6 depicts a surgical system 200 in the context of an operating room (OR) with a surgical robot 202 for implementing the embodiments of the method of FIG. 4. The surgical system 200 of FIG. 5 includes a surgical robot 202, a computing system 204, and an optional tracking system 206. The surgical robot 202 may include a movable base 208, a manipulator arm 210 connected to the base 208, an end-effector 211 located at a distal end 212 of the manipulator arm 210, and a force sensor 214 positioned proximal to the end-effector 211 for sensing forces experienced on the end-effector 211. The base 208 includes a set of wheels 217 to maneuver the base 208, which may be fixed into position using a braking mechanism such as a hydraulic brake. The base 208 may further include an actuator to adjust the height of the manipulator arm 210. The manipulator arm 210 includes various joints and links to manipulate the end-effector 211 in various degrees of freedom. The joints are illustratively prismatic, revolute, spherical, or a combination thereof.

The computing system 204 generally may include a planning computer 216; a device computer 218; a tracking computer 220; and peripheral devices. The planning computer 216, device computer 218, and tracking computer 220 may be separate entities, one-in-the-same, or combinations thereof depending on the surgical system. Further, in some embodiments, a combination of the planning computer 216, the device computer 218, and/or tracking computer 220 are connected via a wired or wireless communication. The peripheral devices allow a user to interface with the surgical system components and may include: one or more user-interfaces, such as a display or monitor 112 for the graphical user interface (GUI); and user-input mechanisms, such as a keyboard 114, mouse 122, pendent 124, joystick 126, foot pedal 128, or the monitor 112 that in some inventive embodiments has touchscreen capabilities. In a particular embodiment, the monitor 112 includes a graphical user interface (GUI) to display the output from the ligament/tendon trial implant graft.

The planning computer 216 contains hardware (e.g., processors, controllers, and/or memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include pre-operative bone data, patient data, registration data including the POSE of a set of points P defined relative to the pre-operative bone data, ligature implant and tunnel position data, trajectory parameters, and/or operational data. The operational data may be a set of instructions for modifying a volume of tissue that is defined relative to the anatomy, such as a set of cutting parameters (e.g., cut paths, velocities) in a cut-file to autonomously modify the volume of bone, a set of virtual boundaries defined to haptically constrain a tool within the defined boundaries to modify the bone, a set of planes or drill holes to drill pins or tunnels in the bone, a graphically navigated set of instructions for modifying the tissue, and the trajectory parameters for robotic insertion of an implant. The operational data specifically includes a cut-file for execution by a surgical robot to autonomously modify the volume of bone, which is advantageous from an accuracy and usability perspective. The surgical plan data generated from the planning computer 216 may be transferred to the device computer 218 and/or tracking computer 220 through a wired or wireless connection in the operating room (OR); or transferred via a non-transient data storage medium (e.g., a compact disc (CD), a portable universal serial bus (USB) drive) if the planning computer 216 is located outside the OR.

In specific embodiments, the computing system 204 does not include a planning computer 216. Rather, the ligament/tendon tunnels are created by aligning the end-effector 211 at a desired trajectory, where the trajectory is defined with the aid of a manual alignment guide. The manual alignment guide may illustratively include the guide described in U.S. Pat. No. 4,739,751. The user may position the alignment guide to designate an optimal trajectory for the bone tunnels. The end-effector 211 (e.g., a drill) is then hand guided to the optimal trajectory as designated by the alignment guide. Once aligned, the motion of the surgical robot 202 is then restricted to only move along the tool axis, which is aligned with the optimal trajectory. Next, in one embodiment, the surgical robot automatically drills the bone tunnels along the optimal trajectory, while in another embodiment, the end-effector 211 is hand-guided by the user along the optimal trajectory. The motion of the robot 202 may be controlled by the device computer 218.

The device computer 218 in some inventive embodiments is housed in the moveable base 208 and contains hardware, software, data and utilities that are preferably dedicated to the operation of the surgical robotic device 202. This may include surgical device control, robotic manipulator control, the processing of kinematic and inverse kinematic data, the execution of registration algorithms, the execution of calibration routines, the execution of operational data (e.g., cut-files, the trajectory parameters), coordinate transformation processing, providing workflow instructions to a user, and utilizing position and orientation (POSE) data from the tracking system 206. In some embodiments, the surgical system 200 includes a mechanical digitizer arm 205 attached to the base 208. The digitizer arm 205 may have its own tracking computer or may be directly connected with the device computer 218. The mechanical digitizer arm 205 may act as a digitizer probe that is assembled to a distal end of the mechanical digitizer arm 205. In other inventive embodiments, the system includes a hand-held digitizer device 202 with a probe tip.

The tracking system 206 may be an optical tracking system that includes two or more optical receivers 207 to detect the position of fiducial markers (e.g., retroreflective spheres, active light emitting diodes (LEDs)) uniquely arranged on rigid bodies. The fiducial markers arranged on a rigid body are collectively referred to as a fiducial marker array (209a, 120a, 120b, 209d), where each fiducial marker array has a unique arrangement of fiducial markers, or a unique transmitting wavelength/frequency if the markers are active LEDs. An example of an optical tracking system is described in U.S. Pat. No. 6,061,644. The tracking system 206 may be built into a surgical light, located on a boom, a stand 234, or built into the walls or ceilings of the OR. The tracking system computer 220 may include tracking hardware, software, data, and utilities to determine the POSE of objects (e.g., bones B, surgical device 202) in a local or global coordinate frame. The POSE of the objects is collectively referred to herein as POSE data, where this POSE data may be communicated to the device computer 218 through a wired or wireless connection. Alternatively, the device computer 218 may determine the POSE data using the position of the fiducial markers detected from the optical receivers 207 directly.

The POSE data is determined using the position data detected from the optical receivers 207 and operations/processes such as image processing, image filtering, triangulation algorithms, geometric relationship processing, registration algorithms, calibration algorithms, and coordinate transformation processing.

The POSE data is used by the computing system 204 during the procedure to update the POSE and/or coordinate transforms of the bone B, the surgical plan, and the surgical robot 202 as the manipulator arm 210 and/or bone(s) (F, T) move during the procedure, such that the surgical robot 202 can accurately execute the surgical plan.

In another inventive embodiment, the surgical system 200 does not include an optical tracking system, but instead employs a mechanical arm 205 that may act as a tracking system 206 as well as a digitizer. If the bone is not tracked, a bone fixation and monitoring system may fix the bone directly to the surgical robot 202 to monitor bone movement as described in U.S. Pat. No. 5,086,401.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A device for measuring mechanical properties of an intended ligament or tendon graft during a surgical procedure, said device comprising:
a sensor body;
a set of temporary fixation securements that terminate a set of opposing sides of said sensor body, said set of temporary fixation securements adapted to attach to at least one of: (i) one or more patient bones, or (ii) permanent fixation hardware assembled to one or more patient bones; and
electronic circuitry in communication with said sensor body, said electronic circuitry generates electric signals in proportion to the mechanical properties experienced by said sensor body.

2. The device of claim 1 wherein said sensor body is modeled to have a form factor that mimics the intended ligament or tendon graft.

3. The device of claim 1 wherein said sensor body is a strain gauge.

4. The device of claim 1 wherein said sensor body is a bonded foil strain gauge.

5. The device of claim 1 wherein said sensory body is a fiber optic strain gauge

6. The device of claim 1 wherein said electronic circuitry is a Wheatstone bridge.

7. The device of claim 1 wherein the sensor body is two strain gauges connected together in a shape that mimics a double bundled ligament or tendon graft.

8. The device of claim 1 wherein the sensor body is a first strain gauge for measuring tension and a second strain gauge for measuring compression.

9. The device of claim 1 wherein the sensor body is three strain gauges, where two strain gauges are tension sensors connected together in a shape that mimics a double bundled ligament or tendon graft, and the remaining strain gauge measures compression.

10. A method for measuring mechanical properties of an intended ligament or tendon graft during a surgical procedure, said method comprising:
placing said sensor body of claim 1 in an intended position of a planned graft;
securing said sensor body with said set of temporary fixation securements to at least one of one or more patient bones that form a movable joint;
exercising the movable joint of the joint bridged relative to said sensor body;
obtaining measurements of the mechanical properties said sensor body is experiencing in response to exercising the movable joint; and
determining whether said sensor body is improperly tensioned, or experiencing impingement based on the obtained measurements.

11. The method of claim 10 further comprising adjusting a length of said sensor body or location of fixation points until the mechanical measurements are satisfactory so as to indicate proper tensioning and acceptable impingement.

12. The method of claim 11 wherein the exercising of the movable joint checks for at least one of fit, stability, or range of motion.

13. The method of claim 11 wherein securing said sensor body to one or more bones comprises attaching said set of temporary fixation securements to permanent fixation hardware installed on one or more patient bones that form a moveable joint.

14. The method of claim 14 further comprising removing said sensor body from the permanent fixation hardware once the mechanical measurements are deemed satisfactory and implanting a permanent graft in the moveable joint using the permanent fixation hardware as fixation points.

15. A computer-assisted surgical system, comprising:
the device of claim 1;
one or more computers with software, said one or more computers receiving and processing said electric signals generated by said electronic circuitry in proportion to the mechanical forces experienced by said sensor body; and
a display to display the output from the one or more computers in real-time.

16. The method of claim 11 wherein the obtained measurements of the mechanical properties said sensor body is experiencing in response to exercising the movable joint are obtained in real time.
